(19) United States
(12) Patent Application Publication
(10) Pub. No.: US 2008/0300554 A1
(43) Pub. Date: Dec. 4, 2008

(54) LUBRICANT FOR MEDICAL DEVICES

(75) Inventor: Masao Yafuso, Lake Foest, CA (US)

Correspondence Address: APPLIED MEDICAL RESOURCES CORPORATION, 22872 Avenida Empresa, Rancho Santa Margarita, CA, 92688, US

(73) Assignee: APPLIED MEDICAL RESOURCES CORPORATION, Rancho Santa Margarita, CA (US)

(21) Appl. No.: 12/130,857

(22) Filed: May 30, 2008

Related U.S. Application Data
(60) Provisional application No. 60/940,888, filed on May 30, 2007.

(51) Int. Cl. A61M 25/00; A61K 9/00

(52) U.S. Cl. 604/265; 523/121

(57) ABSTRACT

A formulation and method for providing medical devices with a lubricious and non-toxic coating Medical devices having a lubricious, non-toxic coating.

This is a non-provisional application claiming the priority of provisional application Ser. No. 60/940,888, filed on May 30, 2007, entitled “Non-Toxic Lubricant for Medical Devices,” which is fully incorporated herein by reference.

### BACKGROUND

Surgical access devices of the prior art typically include a sheath having an outside diameter and an inside diameter. An obturator or dilator is inserted into the sheath to facilitate introduction of the sheath into the body conduit Once the sheath is positioned, the obturator is removed leaving a working channel for surgical instrumentation.

A common problem which occurs in sheath placement is friction or adhesion between the sheath and the dilator. This can be seen in placing other medical devices as well. For example, friction can occur between a catheter and a guide wire or between a guide wire and a stent. Such friction may increase the difficulty of insertion and result in discomfort or damage to the patient, particularly where the device must traverse tortuous pathways in the body Lubricants have been developed to coat medical devices to increase lubricity and thus reduce friction, but these coatings often use undesirable organic solvents.

It would, therefore, be advantageous to develop a radiation sterilizable lubricant coating process for medical devices, and in particular, for urinary tract products, that eliminated the undesirable organic solvents of conventional processes. Preferably, the degree and durability of lubricity should be comparable to the current performance In addition, it is desirable to reduce the tendency for the coating to become “sticky” when allowed to dry after being wet during use.

### SUMMARY

The present invention is directed to a formulation for coating a medical device, the formulation comprising a layering compound and a lubricating compound. The layering compound may be selected from the group consisting of polyethyleneimine (PEI), Tris(2-aminoethyl)amine (TREN), poly(allylamine), putrescine, cadaverine, spermidine, and spermine. Preferably, the layering compound is a cationic polyamine such as PEI.

The lubricating compound may be selected from the group consisting of polyvinylpyrrolidone (PVP), carboxymethylcellulose (CMC), sodium carboxymethylcellulose, hydroxyethyl cellulose (HEC), hydroxypropyl methylcellulose (HPMC), methylcellulose (MC), hydroxyethyl methylcellulose (HEMC), hydroxypropyl cellulose, alginic acids, carrageenans, hyaluronic acids, polyethylene glycol (PEG) and polyethylene oxides (PEO) Preferably, the lubricating compound is PVP

In one embodiment of the present invention, the formulation further comprises a cross-linking agent, preferably a multifunctional epoxide such as ethylene glycol diglycidyl ether (EGDE).

In one embodiment of the present invention, the formulation comprises 0.5% PEI and 10% PVP in isopropanol.

The present invention is also directed to medical devices, such as sheaths, catheters, dilators, and the like, having a lubricious coating, wherein the lubricious coating comprises a layering compound and a lubricating compound.

The present invention is also directed to a method for providing a medical device with a lubricious coating, the method comprising the steps of dipping the device into a solution comprising a layering compound and a lubricating compound, air drying the device, and baking the device at a temperature from about 70° C. to about 90° C. Optionally, the inventive method may also include the step of dipping the coated device into a solution comprising a cross-linking agent.

In one embodiment, the solution comprises PEI and PVP in isopropanol, preferably 0.5% PEI and 1.0% PVP in isopropanol.

In one embodiment, the cross-linking agent comprises EGDE, preferably a 0.1% aqueous solution of EGDE. Other cross-linking agents include glutaraldehyde and polyethyleneglycol diglycidyl

### BRIEF DESCRIPTION OF THE DRAWINGS

FIG. 1 is a graph showing the effect of PVP concentration and temperature on lubricity, using 14-French dilators as a substrate

FIG. 2 is a graph showing the effect of PEI concentration on lubricity, using 14-French dilators as a substrate.

FIG. 3 is a graph showing the PEI concentration effect in ten sequential pulls, with PVP concentration at 1%

FIG. 4 is a graph showing the effect of temperature on lubricity for 0.25% PEI and 1% PVP.

FIG. 5 is a graph showing the effect of temperature on lubricity for 0.5% PEI and 1% PVP.

FIG. 6 is a graph showing the comparative effect of temperature on lubricity at 0 25% PEI and 0.5% PEI, on the tenth pull.

FIG. 7 is a graph showing the effect of time at 81° C. on lubricity for (A) 0.25% PEI and (B) 0.5% PEI, with comparative bar graph shown in (C).

FIG. 8 is a graph showing the effect of room temperature aging on lubricity for (A) 0.25% PEI and (B) 0.5% PEI.

FIG. 9 is a graph showing the effect of PEI concentration on lubricity, before and after baking for 30 minutes at 130° C.

FIG. 10 is a graph showing the effect of PEI molecular weight on lubricity, with and without baking for 15 minutes at 80° C.

FIG. 11 is a graph showing the effect of gamma sterilization on lubricity for (A) 0.25% PEI and (B) 0.5% PEI.

FIG. 12 is a graph comparing the lubricity of current 12-French green sheaths with sheaths coated with 1% PVP, 0.5% PEI.

FIG. 13 is a graph showing lubricity durability by “pull-testing”, comparing products coated with (A) cross-linked PEI/PVP, (B) TS-48, and (C) uncross-linked PEI/PVP

FIG. 14 is a graph showing the set of pull data associated with cytotoxicity data, provided on a more sensitive scale.

FIG. 15 are plots showing random samples tested for lubricity durability and compared with uncross-linked and TS-48 coated production samples.

### DETAILED DESCRIPTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the structures and/or methodologies that are described in the publications which might be used in connection with the presently described invention The publications discussed above and throughout the text are provided solely for their disclosure prior to the filing date of the present application. Nothing herein is to be construed as an admission that the inventors are not entitled to antedate such disclosure by virtue of prior invention.

A single dip coating process was developed that produced a radiation sterilizable lubricant coating for medical devices, which did not require the use of undesireable organic solvents and which provided a high degree and durability of lubricity without becoming sticky when allowed to dry. The ingredients were dissolved in isopropanol to form a stable solution that could be reused continuously, discounting eventual pollution by accumulation of introduced contaminants. The components were fully soluble in isopropanol, but required some dedicated agitation to achieve homogeneity because of the high viscosity of one component and the solid form of the other.

The inventive formulation comprises a “layering” compound, having charged groups (such as amino groups) so as to interact with both the surface of the medical device and a lubricant compound. In one preferred embodiment, this layering compound comprises a cationic polyamine, preferably polyethyleneimine (PEI) although other suitable compounds, such as Tris(2-aminoethyl)amine (TREN), poly(allylamine), putrescine, cadaverine, spermidine, and spermine, for example, will be known to one of skill in the art. The layering compound adheres to the surface of the medical device and interacts with a lubricating compound such as polyvinylpyrrolidone (PVP), carboxymethylcellulose (CMC), sodium carboxymethylcellulose, hydroxyethyl cellulose (HEC), hydroxypropyl methylcellulose (HPMC), methylcellulose (MC), hydroxyethyl methylcellulose (HEMC), hydroxypropyl cellulose, alginic acids, carrageenans, hyaluronic acids, polyethylene glycol (PEG) and polyethylene oxides (PEO), etc., to adhere the lubricating compound to the medical device.

One embodiment of the formulation is as follows:
0.5% polyethyleneimine (PEI, m.wt. 0.6K-1M)
1.0% polyvinylpyrrolidone (PVP, m.wt 120K)

### Isopropanol

One embodiment of the process is as follows:
1 Dip the product in the solution
2 Air dry
3. bake in 81° C. oven for 15 minutes

The formulation has a broad range of tolerance in most parameters. The concentration of the components can be varied by a wide margin and still be effective but data was gathered that shows a broad optimum at the stated concentrations. The baking cycle also shows a wide effective range in time and temperature. This will allow a generous degree of freedom in adapting to manufacturing constraints. A final baking temperature of 81° C. was selected as the benchmark because it was a temperature used in current manufacturing processes. The mechanism for the baking effect has not been determined, but may be some form of condensation reaction between PEI and PVP, promoted at the higher temperatures.

Another variable is the molecular weights of the active components. Both PEI and PVP are commercially available in many molecular weight ranges. PEI was tested to a limited extent at molecular weights of 10K and 70K but no differences were detected when compared with PEI at nominal molecular weights of 0.6K to 1M. Other PVP molecular weights were not tested since the 120K PVP was currently used in production. However it is likely that other molecular weight ranges will work as well.

The cytotoxicity of the PEI/PVP coating was eliminated by immobilizing the PEI by cross-linking the PEI with ethylene glycol diglycidyl ether (EGDE). This was accomplished by a simple dip of the PEI/PVP coated product into a 0.1% aqueous solution of EGDE. In addition, the cross-linking made the coating much more durable with no loss in lubricity. Pull tests showed that the lubricity remained intact even after incubation in phosphate buffered saline (PBS) at 70° C. for 20+ hours. In contrast, the lubricity provided by uncrosslinked coating and the current TS-48 coating degrade considerably after this treatment EGDE itself is cytotoxic but becomes non-cytotoxic once it reacts with PEI.

Experimental results: Test values were generated by a 4-lb force gauge fixture set to record peak value Each sample was subjected to ten sequential pulls after a douse of water before each pull and the peak value recorded. As a general procedure three duplicate samples were tested and averages calculated Occasionally, single readings in a sequence gave anomalously high values. These values were rejected if they were greater than several times the standard deviation of the whole.

a. PEI and PVP Concentration Effects.

It was found that a PEI-PVP solution in isopropanol deposited a uniform lubricious coating on pellathane devices The blue 14f dilator, Applied Medical PN100733203, was selected as representative of the pellathane surfaces to be coated To find an optimum concentration for PVP and PEI, the PVP concentration was varied from 0.25% to 1% while PEI concentration was kept at 1% and the lubricity measured. The results are presented by FIG. 1.

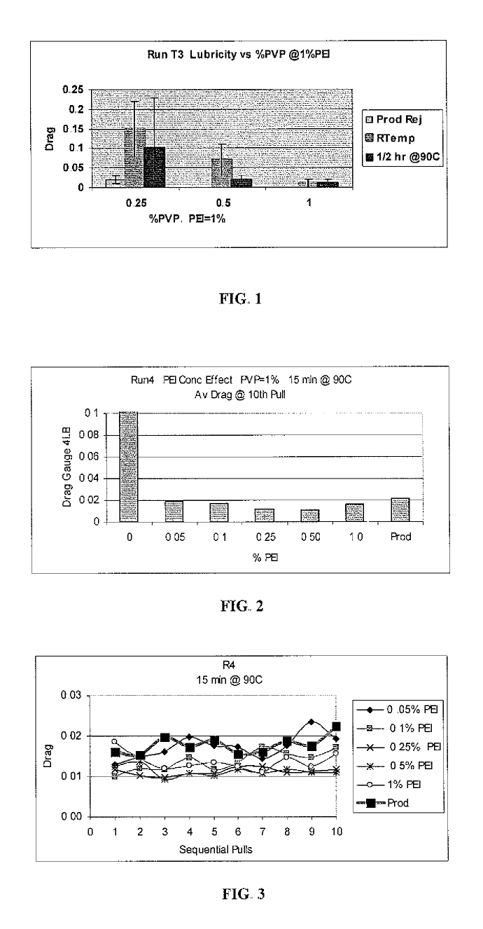

The effect of PEI concentration on lubricity is presented in FIG. 2, which shows the drag at the tenth pull (Note: Value at 0% PEI is off-scale @0.37. A minimum drag occurred @0.25-0.5% PEI). It was assumed that lubricity would be at its worst at the tenth pull. FIG. 2 also shows that PVP alone was not able to provide the necessary lubricity to pellathane surfaces. The reason for this is most probably due to the nature of the dry material and the inability of PVP to adhere to the hydrophobic pellathane surface. A wetting agent was required and PEI served this function.

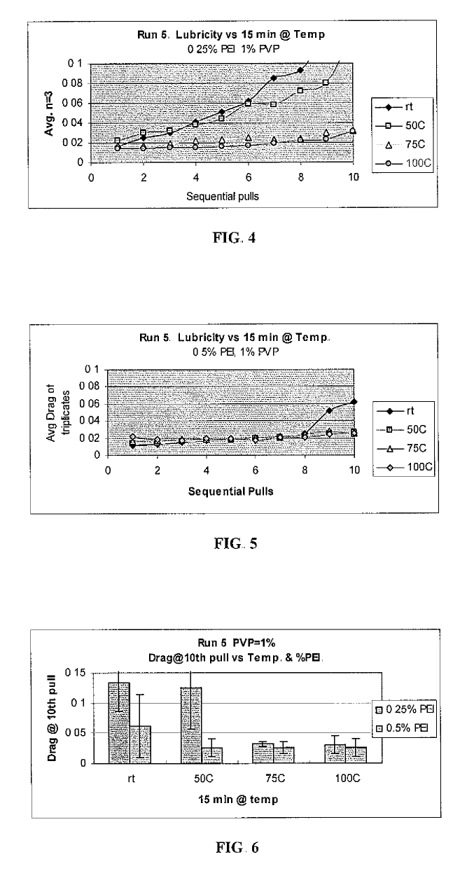

FIG. 3 presents the result of the average of all ten pulls. Note that each point is average of triplicates. 0% PEI is not shown because it is off-scale.

b. Temperature Effects

It was found that some elevated temperature treatment of the coating was very beneficial (see FIGS. 4-6) This effect was most pronounced for low PEI concentrations; in particular, as shown in FIGS. 4 and 6, temperature treatment had dramatic results with 0.25% PEI. Occasionally, at optimum PEI concentrations, samples that were not baked performed equally well. Such results suggested the possibility of a room temperature ageing effect. Such an effect could not be confirmed experimentally leaving these as unexplained anomalies.

As noted above, the temperature effect is more pronounced at low PEI concentrations. At higher concentrations the temperature benefit can be obtained at lower temperatures. Later results suggest that at 1% and higher PEI concentrations, temperatures much above 81° C. will lower lubricity.

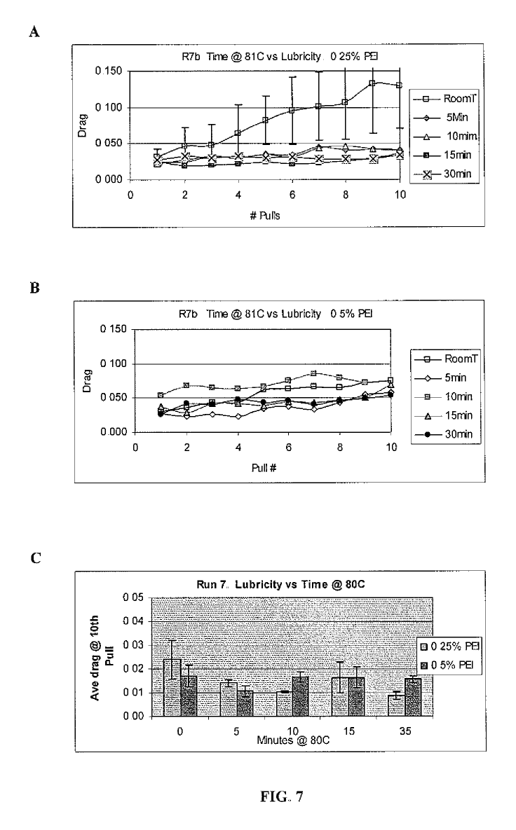

FIG. 7 shows the effect of time at 81° C. on lubricity for both 0.25% and 0.5% PEI. This study indicates that the temperature effect was not very pronounced for PEI concentrations of 0.25% and 0.5%. Other studies showed the existence of a significant temperature effect Based on the cumulative observations, 15 minutes at 81° C. was chosen for a baseline process with the understanding that there was a large safety margin in setting the range of temperature and duration.

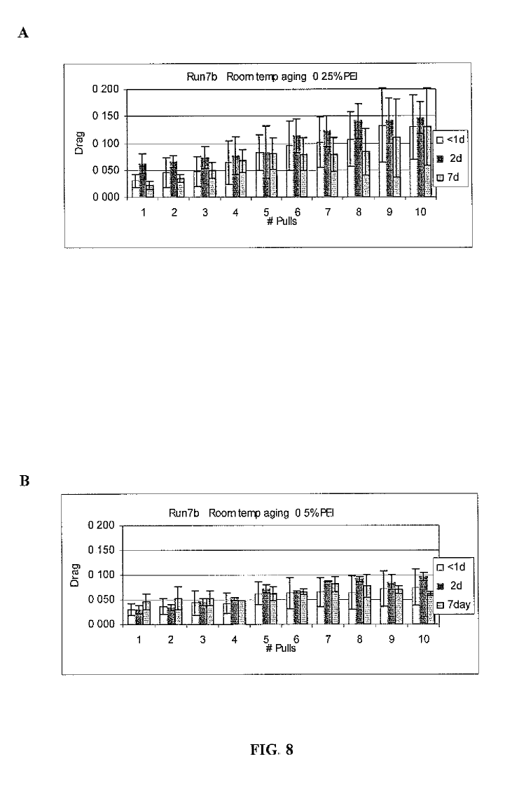

FIG. 8 shows the effect of room temperature aging on lubricity for both 0.25% and 0.5% PEI. These results show very little room temperature ageing effect and could not explain some samples that gave good results without temperature treatment.

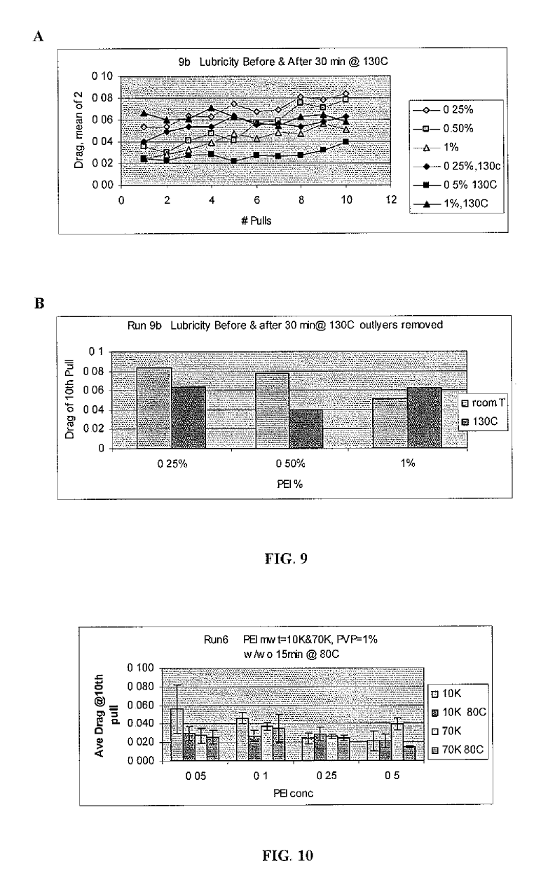

To determine if any chemical instability could be detected visually, test tubes were coated with formulations of PEI concentrations that ranged from 0.05% to 1% and baked at 130° C. for 30 minutes. All formulation formed colorless films that remained colorless after 30 minutes at 130° C., except for the formulation that contained 1% PEI. This coat developed a slight amount of white marbling. To determine the effect of this treatment on lubricity, PEI formulations of 0.25%, 0.5% and 1% PEI in 1% PVP were applied to 14F dilators and tested for lubricity. The results shown in FIG. 9 indicate that too high a temperature will have a deleterious effect on lubricity, especially at 1% PEI. It is anticipated that this effect will be more pronounced with higher in PEI concentrations.

c. PEI Molecular Weight Effects.

Two other molecular weights of PEI (10K and 70K) were available and tested in the lubricant formulations. No significant differences were detected. These results, shown in FIG. 10, indicate that these molecular weights may be considered as alternatives in the inventive formulation.

d. Results of 1×, 2×, and 3× Gamma Sterilizations.

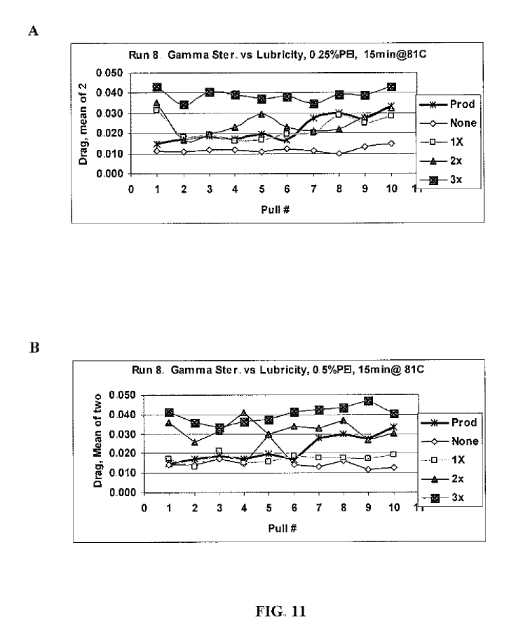

Two sets of 14f dilators were coated with 1% PVP solutions that were 0.25% and 0.5% in PEI respectively. The coated samples were baked for 15 minutes at 81° C. The samples then were divided further into four groups each to be evaluated, before radiation sterilization and after exposure to radiation sterilization for 1×, 2×, and 3×. The results are shown in FIG. 11. There was a nominal loss of lubricity after each radiation cycle but the loss is within tolerable ranges even after three sequential cycle of radiation sterilization.

e. Green Sheaths

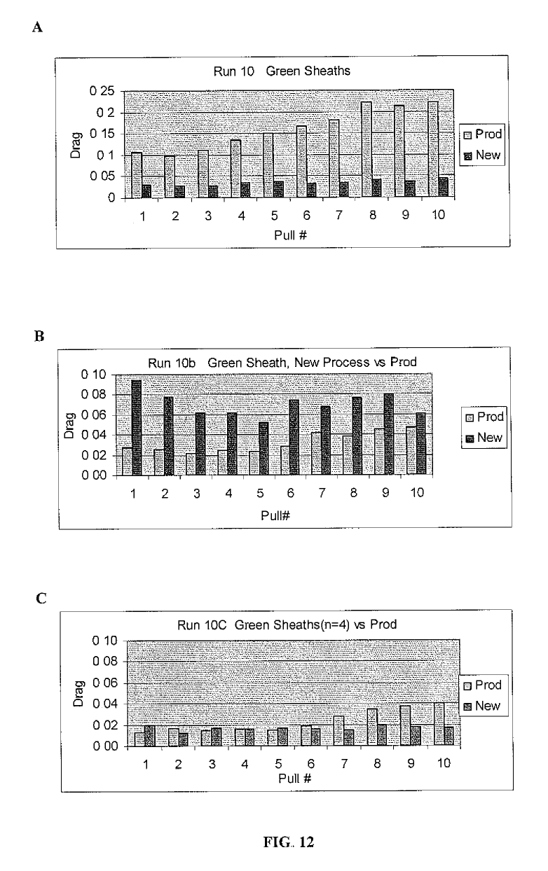

The final formulation developed with dilators were applied to 12f Green sheaths, Applied Medical PN100784302, as a representative ureteral sheath. The tests were repeated three times due to data scattering. The variability was attributed to the limitations of the test fixtures and the results interpreted as averaging out to be equivalent The results are shown in FIG. 12. Note that Run 10 is shown at a different scale than Runs 10b and 10c.

f. Stickiness Test Results

Some conventional sheath-dilator products have a tendency to stick to each other if the mated combination is allowed to dry after wetting. This may occur, for example, if there is an unanticipated delay during a procedure. Mated pairs of green Sheath-dilators were coated with 0.5% PEI, 1% PVP solution, baked for 15 minutes at 81 C, then tested for stickiness as follows: the sheath and dilator were wet separately and then assembled. The dilator was removed from the sheath every five minutes and an estimate of the degradation of coating effectiveness made after each removal. Following each test period, the sheath and dilator were reassembled. After thirty-five minutes, the dilator was removed from the sheath and both components dried over night. The test was then repeated the next day with the same samples.

The new coating exhibited only a few incidences of minor stickiness at the tip. The method and results are shown below in Table 1.

TABLE 1
Stickiness Test (Run 10, Pellathane lubricants)

| | 5:00 | 10:00 | 15:00 | 20:00 | 25:00 | 30:00 | 35:00 |
|---|---|---|---|---|---|---|---|
| Sample | | | | | | | |
| 1 | 100% | 100% | 100% | 95¹% | 95¹% | 95¹% | 90% |
| 2 | 100% | 100% | 100% | 100% | 95¹% | 95¹% | 90% |
| 3 | 100% | 100% | 100% | 100% | 100% | 100% | 95% |
| Day 2 | | | | | | | |
| 1 | 100% | 100% | 95¹% | 95¹% | 95¹% | 95¹% | 95¹% |
| 2 | 100% | 100% | 100% | 100% | 95¹% | 95¹% | 95¹% |
| 3 | 100% | 100% | 100% | 100% | 100% | 100% | 100% |

¹Slight stickiness at tip

There are a variety of water soluble, organic solvent insoluble materials that may have worked as lubricants. However, water-based coatings had the manufacturing disadvantage of long drying times. If this could be tolerated, there are many other candidates.

It is important to note that PEI is a globular polymer. In the event that a more linear PEI might exhibit better properties, such as reduced cytotoxicity, efforts were made to linearize this material by cross-linking with di-epoxides, as discussed below. PEI is the only component in the new formulation with cytotoxic potential The other component, polyvinylpyrrolidone (PVP) is nontoxic.

PEI is present to promote wetability and to provide a physical matrix for PVP, which was the main component of lubricity. If PEI leaches into the toxicity test medium, it can cause a cytotoxic result. Therefore, to eliminate such toxicity, it is preferable to immobilize the PEI. To this end, PEI can be cross-linked ionically or covalently, making it immobile without affecting PVP. Ionic cross-linking can be accomplished with available polyanions while covalent cross-linking can be accomplished with any number of readily available multifunctional chemicals. A partial list of PEI Cross-linkers is provided below in Table 2.

TABLE 2
PEI Cross-linkers

| Covalent Cross-Linkers | Ionic cross-linkers |
|---|---|
| Ethylene glycol diglycidyl | Polyacrylic acid (PAA) |
| ether (EGDE) | |
| Other multi-epoxides | Alginic Acid |
| Glutaraldehyde | Carrageenans |

Assay for Dissolved PEI.

To evaluate the effectiveness of PEI immobilization, a test method was devised for low concentrations of PEI. A sensitive method to detect PEI in solution was available. Ninhydrin, a chemical used widely in Forensics (M M Joullie & T R Thompson, Ninhydrin and Ninhydrin Analogs, Syntheses and Applications) and protein biochemistry provides a sensitive assay for primary amines. Ninhydrin forms a colored product called Ruheman's purple with primary amines. A 2% ninhydrin reagent developed by S. Moore is available from Sigma (N7285-100 ml, Ninhydrin Reagent, 2% solution). However, this reagent deteriorated in the presence of oxygen and presented difficulties in perfecting a reliable assay at the PEI concentrations required and was abandoned.

TABLE 3
Ninhydrin Assay for PEI
Evaluation of 2% Ninhydrin Reagent (Sigma N7285) as a PEI assay
Ninhydrin reagent 2% solution. The following PEI (1 M) solutions in
PBS prepared as standards 5 drops of ninhydrin reagent added to
3cc PEI solutions and color observed

| % PEI | Vol (cc) | color | |
|---|---|---|---|
| 0.01 | 3 | deep blue-black | |
| 0.001 | 3 | light purple | |
| 0.0001 | 3 | faint purple | detectability 1 ppm |

This assay showed promise even without the aid of a spectrometer. However it was found that the sensitivity of the test degraded as the reagent aged and frustrated the development of a reliable assay.

Lacking a direct assay for PEI, an indirect measure of cross-linking effectiveness was selected. This method involved measuring the stability of the cross-linked coating when incubated in PBS at 70° C. for 20 hours. Under these conditions, the PEI/PVP coating was readily extracted into the buffer and lubricity was lost. Even the lubricity of the current TS-48 coating was significantly degraded under this treatment. However, the cross-linked PEI/PVP coating survived this treatment with no loss of lubricity. It was assumed that this durability was only possible if cross-linking had immobilized the PEI into a cross-linked matrix. PVP which cannot participate in this cross-linking was free to diffuse through the matrix to maintain lubricity. This assumption was further validated when the covalently cross-linked coating tested as non toxic.

Cross-Linking Studies.

1. Ionic Cross-Linking

PEI is a highly positively charged ionic compound in solution. It was theorized that it would form an insoluble complex with polyanions and thereby lose any cytotoxicity. Several such polyanions are available. Polyacrylic acid (PAA) is a synthetic polyanion. Alginic acid is a linear polyanionic carbohydrate extract. The carrageenans are nonlinear acidic carbohydrate extracts. Biological extracts have the disadvantage of being potential pyrogen carriers. It may also present immunogenicity problems. Polyacrylic acid did not present such concerns All the anions seemed to demonstrate ionic cross-linking capability but focus was put on polyacrylic acid.

Polyacrylic acid (PAA; Aldrich #306215, Mv=1,250,000) formed gelatinous precipitates with PEI even at PEI concentrations of 0.0001% in phosphate buffered saline, suggesting it would be a good candidate for ionic cross-linking of PEI. When a PEI/PVP coated sheath was dipped into a 0.03% aqueous PAA solution, a gelatinous precipitate formed in the PAA solution. This precipitate in the dip solution increased with the dip-coating of additional sheaths which then deposited on subsequent sheaths leaving gelatinous, uneven deposits on the sheaths, a cosmetic problem. It was apparent that PEI was being extracted into the coating solution during dipping.

TABLE 4
Detection of Leaching of PEI into PAA Bath during Coating
This will create a problem during production by forming
aggregates in coating solution that would require frequent
changing of solution, or the development of a misting system
that avoids reuse of dipping solution.
Method: Dip PEI/PVP coated sheath segments
into PAA bath and observe

| Coating bath | Observations |
|---|---|
| 0.03% PAA, aqueous | fair amount of white particles slowly settling |
| 0.03% PAA 67% IPA | fine cloud, much finer than w/ aqueous and |
| | seems less/dip |
| 0.01% PAA 90% IPA | very fine, light cloud. Much finer than @ 67% |
| | IPA |

Conclusion: PEI readily escapes during coating. What escapes forms precipitates that may cause lumpy deposits. The precipitate size becomes finer as the IPA concentration increases.

PEI from a PVP/PEI coated sheath readily leaches into aqueous or isopropanol baths. PAA and other polyanions form insoluble adducts with PEI immobilizing PEI. These adducts should prevent toxic test results but present manufacturing problems in the form of bath contamination with gels and cosmetically unacceptable gelatinous deposits on products. Alginic acid, sodium salt (AA; Sigma A2158-100 g) did not form a precipitate with PEI, which indicated that it would not be suitable for ionic cross-linking. However it indicated some immobilization of PEI on coated sheaths by Ninhydrin testing of incubation fluid.

i-Carrageenan type 11 (Sigma C-1138) 0.5% aqueous formed a precipitate with equal volume of 0.01% PEI upon standing but showed considerable PEI release with Ninhydrin testing K-Carrageenan (Sigma C-1263) also didn't show any promise.

2. Covalent Cross-Linking of PEI: Ethylene Glycol Diglycidyl Ether (EGDE).

Primary amines react readily with epoxides to form a secondary amine alcohol. Therefore, a multifunctional epoxide has the capability of cross-linking PEI into an immobile matrix EGDE (Sigma Chemical, ethylene glycol diglycidyl ether, E27203 50% technical grade) is a diepoxide with this capability. EGDE is readily soluble in water and isopropanol and can be applied to a coated sheath by a simple dip into an EGDE solution. Effectiveness of EGDE as a cross-linker of PEI was evaluated at 0.1% and 0.5% in aqueous and isopropanol solutions. Evidence of cross-linking was determined by the durability of lubricity after incubation in phosphate buffered saline (PBS) for 20+ hours at 70° C.

Samples were prepared for cytotoxicity testing after cross-linking. Sample cohorts were exposed to 1×, 2× and 3× gamma sterilization after cross-linking. The 1× sterilized samples were sent for cytotoxicity testing. Preparation details are given below in Table 5.

TABLE 5
Preparation for radiation sterilization and cytotoxicity testing
Coat 6. Preparation for radiation sterilization and cytotoxicity testing

| | |
|---|---|
| Coat 45 green sheaths with 0.5% PEI (1M)/1% | Bake 15 min @ 70 C. |
| PVP (120) coating prepared by production. | |
| Coat 6A Coat 15 with 0.10% aqueous EGDE | Dry 15 min @ 70 C. |
| (Aldrich E272030). | |
| Coat 6B Coat 15 with 0.5% aqueous EGDE | Dry 15 min @ 70 C. |
| Coat 6C Coat 15 with 0.10% EGDE in IPA | Dry 15 min @ 70 C. |
| Coat 6A1, 6B1, 6C1, 4 ea. Gamma sterilize 1x | |
| Coat 6A2, 6B2, 6C2, 4 ea. Gamma sterilize 2x | |
| Coat 6A3, 6B3, 6C3, 4 ea. Gamma sterilize 3x | |
| Coat 6A, 6B, 6C 3 ea. No gamma sterilization | |

Cytotoxicity results are given below in Table 6.

TABLE 6
Cytotoxicity Scores

SCORES

| Code | 24 hrs | 48 hrs | 72 hrs | Results |
|---|---|---|---|---|
| 6A1 | 0 | 0 | 0 | Pass |
| 6B1 | 1 | 2 | 3 | Fail |
| 6C1 | 2 | 3 | 3 | Fail |

Cross-linking with 0.1% aqueous EGDE gave successful results. Surprisingly, coating with a 0.5% EGDE and 0.1% EGDE in isopropanol failed. This was interpreted to mean that the latter coating solutions left unreacted EGDE, which itself is cytotoxic. It was considered unlikely that cross-linking was less effective in the failed cases and that PEI was causing the cytotoxic effect. This was supported by lubricity durability of those samples.

Lubricity Durability.

This test consisted of incubation of 6 inch segments of the coated sheaths in phosphate buffered saline (PBS) in 18×150 mm test tubes at 70° C. for 20-24 hours. The samples were then tested for lubricity with the Imada Digital Force Gauge in the pull test fixture pulling at 10 inches/minute Each sample was subjected to five sequential pulls and 20-30 data points (20-30 seconds) per pull per sample were recorded and plotted. Some tests were run on 3 inch segments. These samples gave erratic results and were considered too short for this test.

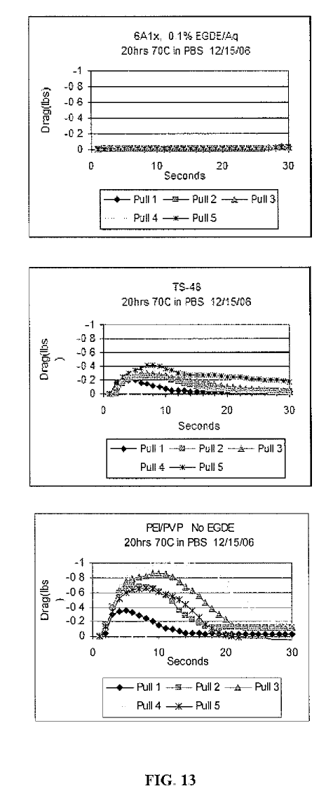

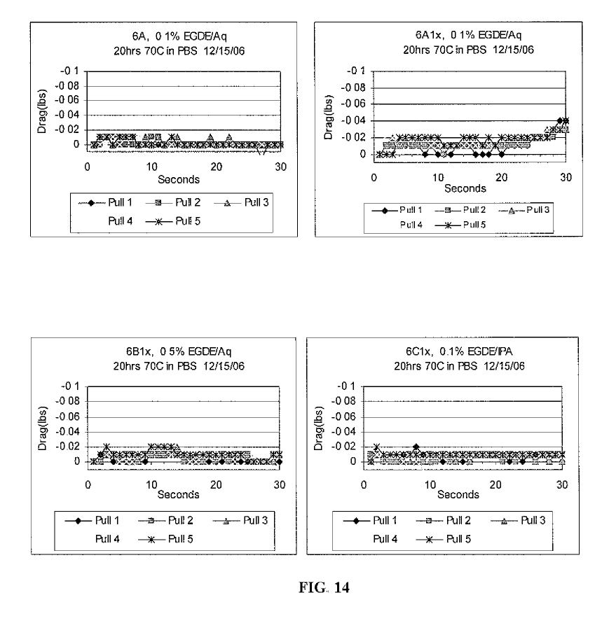

Graphs of lubricity durability by “pull-testing” are shown in FIG. 13 Samples of the current production coating, TS-48, and uncross-linked PEI/PVP coating were included for comparison The data are plotted at a large scale to emphasize the relative difference between cross-linked, uncross-linked and current product TS-48.

The set of pull data associated with the cytotoxicity data is provided in FIG. 14 at a more sensitive scale. The unsterilized sample 6A is included to gauge possible radiation sterilization effects. Note the different in scale

Other Covalent Cross-Linkers.

Glutaraldehyde and polyethyleneglycol diglycidyl ether (Aldrich 475695-500 ml) were screened for cross-link effectiveness and showed lubricity durability (data not shown).

Reusability of 0.1% Aqueous EGDE Coating Solution.

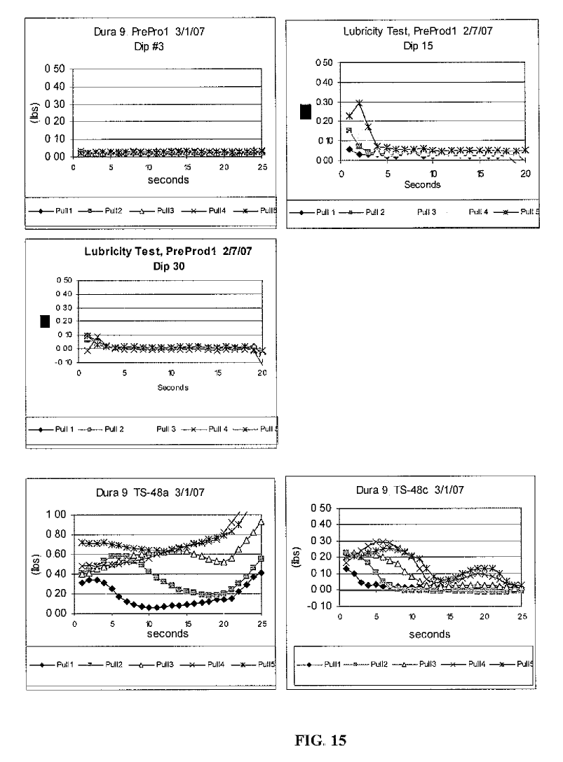

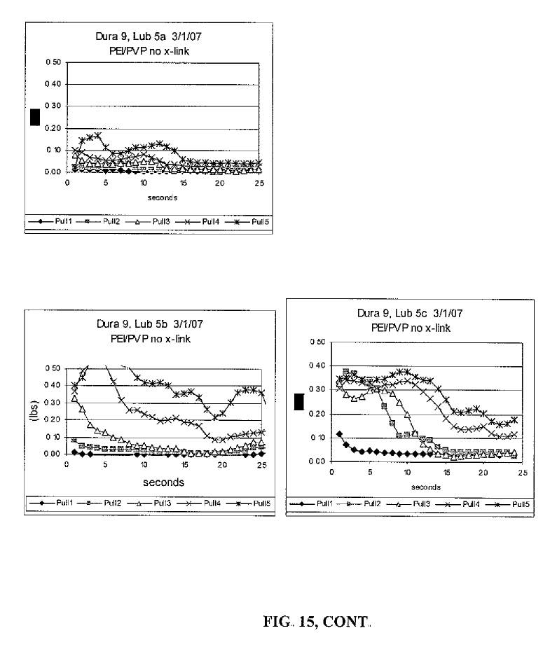

Since a typical shop order consists of 270 units that will be coated nine at a time, or 30 total coating dips, it was necessary to determine how long a single EGDE bath could be used before it became ineffective. The main concern was that PEI will leach into the coating solution and consume the EGDE to exhaustion. To determine if a single solution could be reused for an entire shop order, a preproduction run was made The dipping fixture was limited to three reservoirs and 30 sets of 3 were dip coated sequentially without further change or replenishment of solution. Each sample was labeled by its dip number. Samples were radiation sterilized 1× and samples from dip number 1, 16, and 29 were sent for cytotoxicity testing Each sample passed with a score of 0/0/0 for 24 hrs, 48 hrs and 72 hours in the MEM elution test on confluent mouse fibroblasts Random samples were also tested for lubricity durability, and again compared with uncross-linked and TS-48 coated production samples. Plots are provided in FIG. 15.

It is hypothesized that the lubricity durability of the present invention arises because the cross-linked PEI forms a stable matrix through which the PVP lubricant can diffuse only slowly. This will provide longer lasting lubricity which may be of significant value in longer indwelling products. The cross-linking process should be directly transferable to all medical products benefiting from lubrication.

This is of particular importance in urinary tract products. It has been reported that urinary tract infections account for 30% of all nocosomial infections, most of which are associated with urinary catheters (Dixon G., Surgery 20 179-185 (2002), quoted by Ebrey et al, “Biofilms and Hospital-Acquired Infections” in Microbial Biofilms, Ghannoum & O'Toole, editors, ASM Press 2004). The risk of infection of urinary catheterization has been estimated to increase by 5% for each day the catheter is in place Similarly microbial colonization of ureteral stents have been found to be as high as 44% (Paick et al, Urology 2003:214-217 (2003); characterization of bacterial colonization and urinary tract infection after indwelling of double-J ureteral stent). Bacterial colonization resulting in drug resistant biofilm formation leads to serious clinical complications including urinary encrustations (Tenke P et al., World J Urol. 24 13-20 (2006). The Role of Biofilm Infection in Urology). Prevention of microbial colonization has been cited as a major unsolved problem associated with urinary products. The lubricant formulation if the present invention should provide a useful base from which to address this unsolved problem, in that a slow release lubricant coating can also serve as a reservoir for a slow release antimicrobial activity and ameliorate to some degree this important problem.

Although the present invention has been described in certain specific aspects, many additional modifications and variations would be apparent to those skilled in the art. It is therefore to be understood that the present invention may be practiced otherwise than specifically described, including various changes in the size, shape and materials, without departing from the scope and spirit of the present invention. Thus, embodiments of the present invention should be considered in all respects as illustrative and not restrictive. Also, all the examples provided throughout the entire description should be considered in all respects as illustrative and not restrictive. The scope of the invention is, therefore, indicated by the following claims rather than by the foregoing description. All changes, modifications, and variations coming within the meaning and range of equivalency of the claims are to be considered within their scope

1. A formulation for coating a medical device, the formulation comprising a layering compound and a lubricating compound.

2. The formulation of claim 1, wherein the layering compound is selected from the group consisting of polyethyleneimine (PEI), Tris(2-aminoethyl)amine (TREN), poly(allylamine), putrescine, cadaverine, spermidine, and spermine.

3. The formulation of claim 1, wherein the layering compound is a cationic polyamine.

4. The formulation of claim 3, wherein the cationic polyamine is PEI.

5. The formulation of claim 1, wherein the lubricating compound is selected from the group consisting of polyvinylpyrrolidone (PVP), carboxymethylcellulose (CMC), sodium carboxymethylcellulose, hydroxyethyl cellulose (HEC), hydroxypropyl methylcellulose (HPMC), methylcellulose (MC), hydroxyethyl methylcellulose (HEMC), hydroxypropyl cellulose, alginic acids, carrageenans, hyaluronic acids, polyethylene glycol (PEG) and polyethylene oxides (PEO).

6. The formulation of claim 1, wherein the lubricating compound is PVP.

7. The formulation of claim 1, wherein the layering compound is PEI and the lubricating compound is PVP.

8. The formulation of claim 1, further comprising a cross-linking agent.

9. The formulation of claim 8, wherein the cross-linking agent is a multifunctional expoxide.

10. The formulation of claim 8, wherein the cross-linking agent is ethylene glycol diglycidyl ether (EGDE).

11. A formulation for coating a medical device, the formulation comprising 0.5% PEI and 1.0% PVP in isopropanol.

12. A medical device having a lubricious coating, wherein the lubricious coating comprises a layering compound and a lubricating compound.

13. The medical device of claim 12, wherein the device is a sheath.

14. The medical device of claim 12, wherein the device is a catheter.

15. The medical device of claim 12, wherein the device is a dilator.

16. The medical device of claim 12, wherein the layering compound is selected from the group consisting of polyethyleneimine (PEI), Tris(2-aminoethyl)amine (TREN), poly(allylamine), putrescine, cadaverine, spermidine, and spermine.

17. The medical device of claim 12, wherein the layering compound is a cationic polyamine.

18. The medical device of claim 12, wherein the cationic polyamine is PEI.

19. The medical device of claim 12, wherein the lubricating compound is selected from the group consisting of polyvinylpyrrolidone (PVP), carboxymethylcellulose (CMC), sodium carboxymethylcellulose, hydroxyethyl cellulose (HEC), hydroxypropyl methylcellulose (HPMC), methylcellulose (MC), hydroxyethyl methylcellulose (HEMC), hydroxypropyl cellulose, alginic acids, carrageenans, hyaluronic acids, polyethylene glycol (PEG) and polyethylene oxides (PEO).

20. The medical device of claim 12, wherein the lubricating compound is PVP.

21. The medical device of claim 12, wherein the layering compound is PEI and the lubricating compound is PVP.

22. The medical device of claim 12, further comprising a cross-linking agent.

23. The medical device of claim 22, wherein the cross-linking agent is a multifunctional epoxide.

24. The medical device of claim 22, wherein the cross-linking agent is ethylene glycol diglycidyl ether (EGDE).

25. A method for providing a medical device with a lubricious coating, the method comprising the steps of dipping the device into a solution comprising a layering compound and a lubricating compound, air drying the device, and baking the device at a temperature from about 70° C. to about 90° C.

26. The method of claim 25, further comprising the step of dipping the coated device into a solution comprising a cross-linking agent.

27. The method of claim 25, wherein the solution comprises PEI and PVP in isopropanol.

28. The method of claim 27, wherein the solution comprises 0 5% PEI and 1.0% PVP in isopropanol.

29. The method of claim 26, wherein the cross-linking agent is selected from the group consisting of EGDE, glutaraldehyde and polyethyleneglycol diglycidyl.

30. The method of claim 29, wherein the cross-linking agent comprises a 0.1% aqueous solution of EGDE.
